(19) United States
(12) Patent Application Publication
(10) Pub. No.: US 2025/0261852 A1
(43) Pub. Date: Aug. 21, 2025

(54) INSTRUMENTED CONTACT LENS, AND ASSOCIATED DEVICE FOR MEASURING REFRACTIVE ERROR AND/OR ACCOMMODATION

(71) Applicant: INSTITUT MINES TELECOM, PALAISEAU (FR)

(72) Inventors: Vincent NOURRIT, BREST (FR); Jean-Louis DE BOUGRENET DE LA TOCNAYE, GUILERS (FR)

(21) Appl. No.: 18/857,359

(22) PCT Filed: Apr. 18, 2023
(86) PCT No.: PCT/EP2023/059963

§ 371 Date: Oct. 16, 2024

(30) Foreign Application Priority Data
Apr. 21, 2022 (FR) 2203684

(51) Int. Cl. A61B 3/125; A61B 3/00; A61B 3/028; A61B 3/10

(52) U.S. Cl. CPC A61B 3/125; A61B 3/0008; A61B 3/028; A61B 3/1015

(57) ABSTRACT

A contact lens (1), intended to be worn on an individual's eye, for measuring refractive error and/or the accommodation of the eye, includes a membrane (10) suitable for transparently covering the pupil and for covering the iris of the eye and preferably at least part of the sclera. The contact lens has at least one source of illumination (11, 12) encapsulated in the membrane, the source(s) of illumination being suitable for emitting two light beams or cones (F1, F2), the divergence of which is controlled relative to the axis of the lens, one of the two beams being intended to be directed towards the inside of the membrane, in the direction of the crystalline lens and/or the retina of the eye, so as to create a source point on the retina, while the other of the two beams is intended to be directed towards the outside of the membrane in a direction opposite to the eye.

### TECHNICAL FIELD

The present invention relates to an instrumented contact lens and also to an associated autorefractor.

The lens according to the invention may be a completely autonomous system and installed on at least one eye of an individual.

The invention aims in particular to automatically measure the refractive error of an individual.

### PRIOR ART

An autorefractor is a device that makes it possible to measure non-invasively and objectively the vision correction, also known as refractive error, that a patient requires. This measurement is in general used as a starting point for prescription of spectacles or contact lenses.

Existing autorefractors may be classified into two broad categories: conventional autorefractors and aberrometers.

Conventional autorefractors only allow aberrations of lower orders (sphere, cylinder and axis) to be measured, while aberrometers measure both the lower orders and higher orders (spherical aberration, coma, trefoil, etc.).

A second difference is that conventional autorefractors generally measure the power of the eye in a region of the pupil that is small and central compared to the region measured by aberrometers.

Autorefractors in general use an infrared source directed toward the retina. The measurement of refractive error is based on one or more measurements of light reflected by the retina. For example, if the source emits a cone of light, the autorefractor may determine when a patient's eye is focusing light correctly based on the size and shape of the ring on the retina. The instrument modifies vergence until the image is clear. The process is repeated in at least three meridians of the eye and the autorefractor computes the refraction of the eye, sphere, cylinder and axis. The measurement may also be based on contrast optimization, or the so-called Scheiner principle, deflection of a beam, etc.

In the case of an aberrometer, a source spot is formed on the retina, and the reflected wavefront is analyzed by an analyzer, usually a Shack-Hartmann wavefront sensor (SHWFS).

The instruments formed by conventional autorefractors incorporate, most of the time, a chin rest and always a target on which to focus.

In a certain number of cases, it would be desirable for this measurement to be taken automatically, i.e. without the involvement of a visual health professional such as an ophthalmologist. For example, wearing spectacles may hinder the use of many pieces of modern equipment (telescopic sight, microscope, augmented-reality glasses, virtual-reality headset, etc.). An automatic measurement of refractive error would make it possible to automatically correct it without being constrained by modern equipment, for example when the user raises a telescopic sight to her or his eyes, uses a microscope objective, etc. This would also make it possible to envision making measurement of refractive error an ambulatory test.

Thus, if one of these pieces of equipment incorporated an autorefractor, its optics could be automatically adjusted to the sight of each individual, thus allowing her or him to achieve a perfect image without said individual being obliged to wear corrective spectacles.

One difficulty with such incorporation is that the quality of the measurement requires good alignment of the visual axis of the individual with that of the autorefractor, limiting her or his mobility.

There is therefore a need to further improve existing autorefractors, particularly in order to allow them to be incorporated into modern equipment (telescopic sight, microscope, augmented-reality glasses, virtual-reality headset, etc.), while ensuring good alignment of the visual axis with that of an individual wearing/employing the equipment, without constraining the mobility of the individual, and preferably in a potentially ambulatory context of use.

The aim of the invention is to at least partially meet this need.

### SUMMARY OF THE INVENTION

To this end, one subject of the invention is a contact lens intended to be worn by an eye of the individual, to measure refractive error and/or the accommodation of the eye, comprising:
a membrane configured to transparently cover the pupil and to cover the iris of the eye and preferably at least partially the sclera;
at least one source of illumination encapsulated in the membrane, the one or more sources of illumination being configured to emit two cones or beams of light the divergence of which is controlled with respect to the axis of the lens, one of the two beams being intended to be directed toward the interior of the membrane in the direction of the crystalline lens and/or retina of the eye so as to create a source spot on the retina, while the other of the two beams is intended to be directed toward the exterior of the membrane in a direction away from the eye.

According to one advantageous embodiment, the contact lens comprises at least two separate sources of illumination namely one for emitting the beam intended to be directed toward the interior of the membrane in the direction of the crystalline lens and/or retina of the eye and the other for emitting the beam intended to be directed toward the exterior of the membrane in a direction away from the eye.

According to another advantageous embodiment, the contact lens comprises at least one diffractive optical element, encapsulated in the membrane and configured to receive the light beam emitted by the one or more sources of illumination that is directed toward the interior of the membrane and to diffract said beam in the direction of the crystalline lens and/or retina of the eye and to create at least two separate source spots on the retina so as to generate interference between the beams reflected by the retina.

According to another advantageous embodiment, the contact lens comprises at least one other diffractive optical element, encapsulated in the membrane and configured to receive the light beam emitted by the one or more sources of illumination that is directed toward the exterior of the membrane and to collimate said beam or project a target or reticle, such as a grid, so as to allow the orientation of the gaze, such as cyclo-torsions, to be computed or one or more detectors arranged on the periphery of the eye to be addressed.

Preferably, the sources of illumination emit in the infrared.

In one advantageous variant, the one or more sources of illumination are one or more light-emitting diodes (LEDs), or one or more vertical-cavity surface-emitting lasers (VCSELs) or one or more edge-emitting laser diodes. According to this variant, the one or more LEDs or VCSELs are advantageously each provided with an optical device for shaping their beams.

According to one advantageous embodiment, the contact lens comprises:
at least one interface for collecting and supplying the sources of illumination with electrical energy, from the exterior of the lens;
at least one electronic circuit configured to activate the sources via the interface.

According to this embodiment and one advantageous variant of embodiment, the contact lens comprises a battery encapsulated in the membrane and connected to the interface, the battery being configured to be recharged via the interface and to supply the sources of illumination and/or optoelectronic functions associated with the sources of illumination electrically, the electronic circuit being configured to activate the sources using the battery.

According to one advantageous variant of embodiment, the interface comprises an antenna configured for the transfer of energy by electromagnetic induction, and a rectifier connected to the antenna with a view to transferring all or some of the energy received by the antenna to the sources, and where appropriate to the battery and/or other optoelectronic functions encapsulated in the membrane. The transfer over a distance of energy by induction, particularly with a view to recharging the battery, is advantageous because it may be done easily and rapidly without an external connection, by means of an antenna integrated into the carrier (spectacle frame, augmented-reality headset) that will be used to detect the beams of the sources of illumination.

Preferably, the rectifier is configured to transfer all or some of the energy received by induction directly to the sources of illumination.

According to one advantageous variant, the antenna may be configured to transmit data wirelessly, in particular via radio frequencies (RF).

Advantageously, at least one of the two sources of illumination forms part of a communication system.

Advantageously, the battery is a deformable accumulator encapsulated in the membrane. It may be a question of the accumulator described and claimed in patent application WO2018/167393 A1. Such an accumulator has the advantage of being of very small dimensions, typically having a surface area of about 0.75 cm². This flexible battery also has the advantageous features of being stretchable and self-repairing so as to be better integrated into the contact lens and of being able to ensure sufficient battery life for operation of the sources of illumination.

The contact lens is preferably a rigid or hybrid (semi-rigid) scleral lens. A scleral lens has the advantage of being more stable on the eye than a conventional contact lens, this being advantageous in such a device installed on the eye. A scleral lens has a larger useful area.

Another subject of the invention is an autorefractor comprising:
at least one contact lens such as described above;
a carrier, intended to be positioned stationary with respect to the face of the individual;
at least one detector that is securely fastened to the carrier, the one or more detectors being configured to detect the position of the beam of illumination of the contact lens that is directed toward the exterior so as to extract therefrom the angle of deflection with respect to normal of the gaze;
at least one sensor, preferably a quadratic sensor, which is securely fastened to the carrier, and which forms part of a refractometer configured to locate the wavefront of the beam reflected by the retina and/or crystalline lens and refracted by the eye so as to measure the refractive error of the eye given the angle of deflection measured by the detector.

The contact lens is therefore coupled to at least one detector, preferably a position sensitive detector (PSD), placed facing the eye, this allowing the angle of deflection with respect to normal of the gaze to be extracted, and to at least one other detector that forms one portion of a refractometer. Instead of a PSD, a camera may very well be used. A PSD has the advantage of being simpler, cheaper and more accurate.

According to a first advantageous embodiment, the refractometer is configured to operate as an interferometer configured to measure interference between at least one beam reflected by the retina and refracted by the eye and another illumination beam reflected by the retina.

According to this first embodiment, a plurality of advantageous variants may be envisaged, particularly:
the lens comprises a VCSEL by way of source of illumination, the interferometer being configured to perform back-injection laser interferometry between the beam in the interior of the cavity of the VCSEL and a beam reflected by the retina and refracted by the eye;
the lens comprises a VCSEL by way of source of illumination and a diffractive optical element, the interferometer being configured to measure interference between at least two beams that are diffracted by the diffractive optical element from the same beam emitted by the VCSEL, reflected by the retina and refracted by the eye;
the lens comprises a VCSEL by way of source of illumination and a diffractive optical element, the interferometer being configured to measure interference between at least two beams that are diffracted by the diffractive optical element from the same beam emitted by the VCSEL, one being reflected by the retina and refracted by the eye and the other being reflected by the crystalline lens and refracted by the eye.

According to a second embodiment, the autorefractor comprises, as part of the refractometer, a plurality of detectors that are securely fastened to the carrier and arranged to be distributed around the eye, the lens comprising a plurality of VCSELs by way of source of illumination and a plurality of diffractive optical elements each associated with one of the VCSELs, or a single VCSEL combined with at least one optical component generating a plurality of light beams directed toward said detectors, the refractometer being configured to measure the deflection of the beams emitted sequentially by each of the VSCELs, diffracted by each of the diffractive optical elements, reflected by the retina and refracted by the eye.

According to a third embodiment, the autorefractor comprises, as part of the refractometer, a camera that is securely fastened to the carrier, the lens comprising a VCSEL by way of source of illumination and a diffractive optical element taking the form of a hologram, the camera being configured to analyze the deformation of the pattern of the hologram, reflected by the crystalline lens. This embodiment is suitable for measuring the accommodation of an eye.

The carrier of the PSD may be a mount, intended to be worn on the face of the individual, such as a spectacle frame or an augmented-reality headset or a head-up display (HUD).

According to a first variant, the system comprises a single PSD, intended to be arranged facing the eye, the PSD being transparent in the visible and sensitive in the near infrared (NIR), the sources of illumination of the contact lens emitting in the near infrared.

According to a second variant, the system comprises two PSDs, intended to be arranged on the periphery of the eye, substantially in a plane facing the eye, so as to cover the range of angular variation in position of the eye, the PSDs being arranged so as not to obstruct the vision of the individual.

Thus, the invention essentially consists of a contact lens the membrane of which incorporates/encapsulates at least one source of illumination, preferably a VCSEL, that sends at least one beam toward the exterior of the eye, with a view to determining the gaze direction, and at least one beam for internal illumination of the eye, i.e. of the crystalline lens and/or retina. The light beam reflected by the retina is collected by a detector and analyzed by a suitable device.

The fact that the lens according to the invention makes it possible to determine the gaze direction via the beam directed toward the exterior of the eye makes it possible to avoid a need for a target to focus on as found in autorefractors or aberrometers according to the prior art, this leaving the individual the refractive error of which is measured entirely free to move. The refractive measurement with a lens according to the invention may be made without the voluntary participation of the individual.

In other words, the invention is a lens in which a combination of light beams directed toward the exterior of the eye and toward the interior in the direction of the crystalline lens and/or retina allows the wavefront reflected by the crystalline lens and/or retina and refracted by the human visual system to be located, so as to realign/align the latter and the refractometer.

By virtue of the contact lens according to the invention, a major drawback of prior-art instruments (autorefractors, aberrometers), which constrain an individual to remain immobile in front of a target on which to focus and provided for this purpose, with a view to ensuring alignment of her or his visual axis, is overcome. Specifically, the various known techniques for objectively measuring refractive error (contrast optimization, Scheiner principle, beam deflection, aberrometry, etc.) are mostly based on recording the reflection of light from an infrared source from the retina and require precise alignment between the infrared source, eye and detector. Accommodation may be computed based on variations around the refractive error or by following similar methods but using light reflected by the crystalline lens.

With a contact lens according to the invention, the source of illumination of an autorefractor is automatically aligned with the eye because the gaze direction is known, via the beam emitted from the lens, at the time of measurement of the refractive error.

Other advantages and features of the invention will become more clearly apparent on reading the detailed description of examples of implementation of the invention, which is given by way of illustration and non-limitingly with reference to the following figures.

### BRIEF DESCRIPTION OF THE DRAWINGS

FIG. 1 is a schematic cross-sectional view of a contact lens according to the invention according to a first embodiment for measuring the refractive error of an eye of an individual.

FIG. 2 is a schematic view of a contact lens according to the invention in cross section according to one variant of FIG. 1.

FIG. 3 is a schematic view of a contact lens according to another variant of FIG. 1.

FIGS. 4 and 4A are cross-sectional and front views of a contact lens according to the invention according to a second embodiment with the associated detectors on a carrier around the eye, respectively.

FIG. 5 is a schematic cross-sectional view of a contact lens according to the invention according to a third embodiment for measuring the accommodation of an eye of an individual.

FIG. 6 is an overview showing the operation of an electromagnetic-induction-based recharging device allowing the deformable battery encapsulated in a contact lens according to the invention to be recharged.

FIGS. 7A and 7B are views showing successive steps of production of a contact lens according to the invention.

### DETAILED DESCRIPTION

It will be noted that the various elements according to the invention have been shown solely for the sake of clarity and that they are not necessarily to scale.

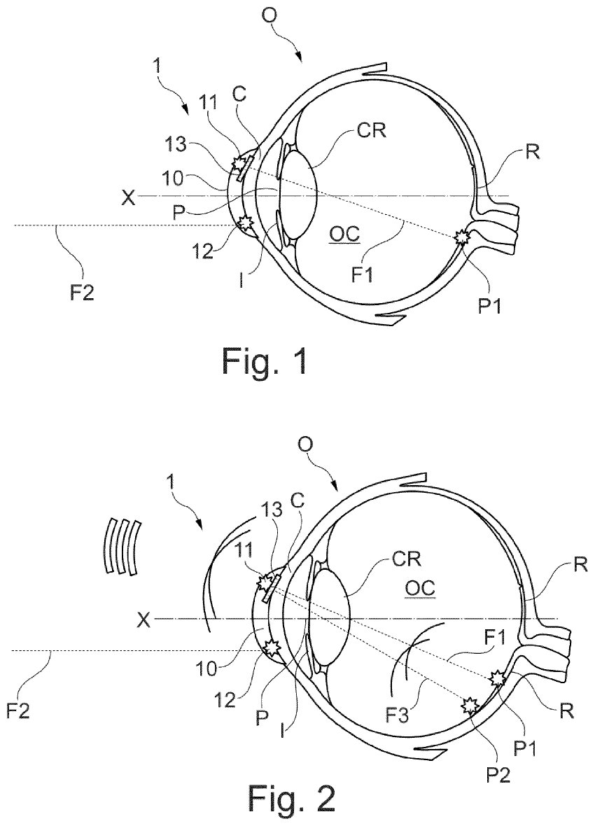

FIG. 1 shows a contact lens 1 according to the invention for measuring the refractive error and/or accommodation of the eye of an individual.

The contact lens 1 is configured to be applied to an eye O of an individual having an optical axis X.

The eye O first has a cornea C, which takes the form of a spherical cap at the interface with ambient air. The eye O has an iris I that is pierced in its center by a circular aperture called the pupil P, through which light is transmitted. The iris I expands or contracts depending on luminous intensity. The eye O also has a crystalline lens CR formed by a fibrous, transparent and flexible disc, to focus the incident light received through the pupil P. Behind the crystalline lens CR, on the other side of an ocular cavity OC, the eye O has a retina R formed from sensory cells including cones for day vision and rods for night vision. As may be seen in FIG. 1, the cornea C, the pupil P and the crystalline lens CR are substantially centered on the optical axis X.

The contact lens 1, which is preferably a rigid or hybrid scleral lens, bears, through encapsulation in its membrane 10, two sources of illumination 11, 12. The membrane 10 is intended to be worn on the eye O of an individual, the anatomy of which has just been described.

These sources may be light-emitting diodes (LEDs) or vertical-cavity surface-emitting lasers (VCSELs) or edge-emitting laser diodes. The light emitted in the infrared, by these sources 11, 12, may be coherent (VCSEL) or weakly coherent (LED). Preferably, the sources 11, 12 are VCSELs.

It may be envisioned to shape the sources 11, 12 (such as for example diodes of elliptical shape) or provide each of the sources with a shaping optical device so that each light beam takes the form of a thin pencil beam in the detection zone, as described below.

The contact lens may incorporate within its membrane 10 a rechargeable stand-alone battery that powers the sources. This battery is advantageously a deformable accumulator such as described and claimed in patent application WO2018/167393A1.

The shape of the membrane 10 is that of a disk that is curved about a central axis, with a concave rear face and a convex front face. The rear face has a shape complementary to the cornea C so that it may be pressed against it or at least cover the latter in a preferred position of the contact lens 1 illustrated in FIG. 1. In the case where the lens is a scleral lens, in the preferred position, there is strictly speaking no direct contact between the lens and the cornea C. The lens bears against the sclera and there is a reservoir of lacrimal fluid between the lens and the cornea C. In this preferred position, the contact lens 1 is centered on the optical axis X, so that the central axis of the membrane 10 is substantially coincident with the optical axis X.

The transparent membrane in contact with the cornea C is preferably made of a biocompatible material, for example one based on silicone hydrogel or HEMA (acronym of hydroxy ethyl methacrylate). It may be any other suitable biocompatible material, as for example described in publication [1].

When the contact lens 1 is being worn on an eye (O) of the individual, each source of illumination 11, 12 may emit a cone or beam of light F1, F2.

According to the invention, the beam F1 illuminates the crystalline lens and/or the retina R of the eye, while simultaneously the beam F2 illuminates toward the exterior in order to achieve an optical indication of direction that allows the gaze direction to be determined. It is advantageously possible to produce and detect the beam F2 as described in patent application WO2020/212394.

The light beam which is reflected by the crystalline lens and/or retina and analyzed by a refractometer, part of which is formed by a beam detector which may, depending on the configuration, be installed in the lens and/or in a carrier that remains stationary relative to the eye of the individual. Knowledge of the gaze direction makes it possible to compensate for the variable alignment of the eye with respect to the position sensitive detector (PSD).

Generally, none of the sources of illumination 11, 12 installed in the lens 1 and none of the detectors installed in the lens and/or on a carrier and making it possible either to determine the gaze direction or to detect the beam reflected by the crystalline lens and/or retina, block the sight of the individual.

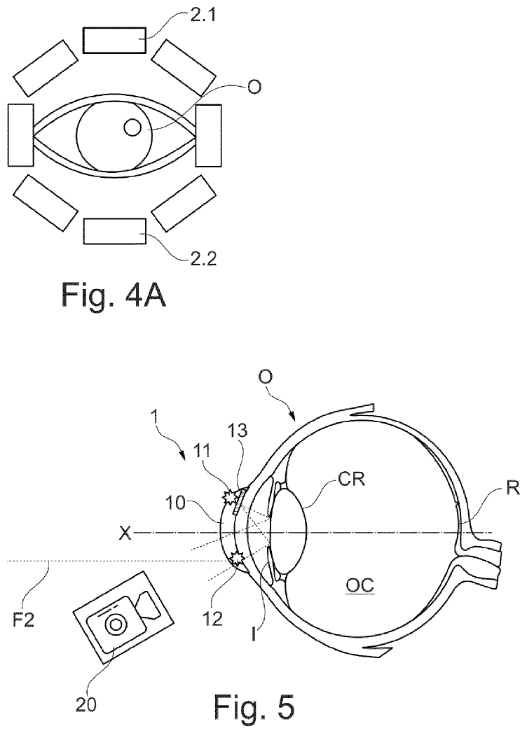

The one or more detectors for detecting the beams F1, F2 are advantageously placed around the eye, preferably on a carrier worn by the individual, such as spectacles, as schematically shown in FIG. 4A.

A number of embodiments of a contact lens 1 according to the invention may be envisioned depending on the measuring method employed by the refractometer.

FIG. 1 shows an embodiment for taking an interferometric measurement by means of one and the same source of illumination 11, namely a VCSEL. This laser emits the beam F1 toward the retina R, creating a source spot P1.

This beam F1 is back-injected into the VCSEL 11.

This VCSEL 11 comprises a photodiode integrated into the cavity of the laser.

The measurement of refractive error is based on back-injection laser interferometry.

Thus, part of the reflected beam is injected into the cavity. The laser field within the cavity and the back-injected laser field are in phase or out of phase, thus modulating the output optical power via self-mixing interference. A fraction of the modulated output power is measured by the photodiode, and the phase difference between the beams and therefore the length of the eye may be computed. Based on knowledge of the alignment of the eye obtained through the beam F1 and this computed eye length, the refractive error of the eye is determined.

FIG. 2 illustrates one variant of the interferometric measurement. Here, the VCSEL 11 is associated with a diffractive optical element 13, which diffracts the beam of the laser 11 into two separate beams F1, F3, which create two separate source spots P1, P2 on the retina R. The light reflected by these two spots P1, P2 creates an interference pattern that is able to be detected by one or more sensors around the eye, preferably quadratic sensors 2.1 . . . as shown in FIG. 4A. Based on knowledge of the alignment of the eye obtained through the beam F1 and this measurement of interference between two beams generated by separate source spots P1, P2, the refractive error of the eye is determined.

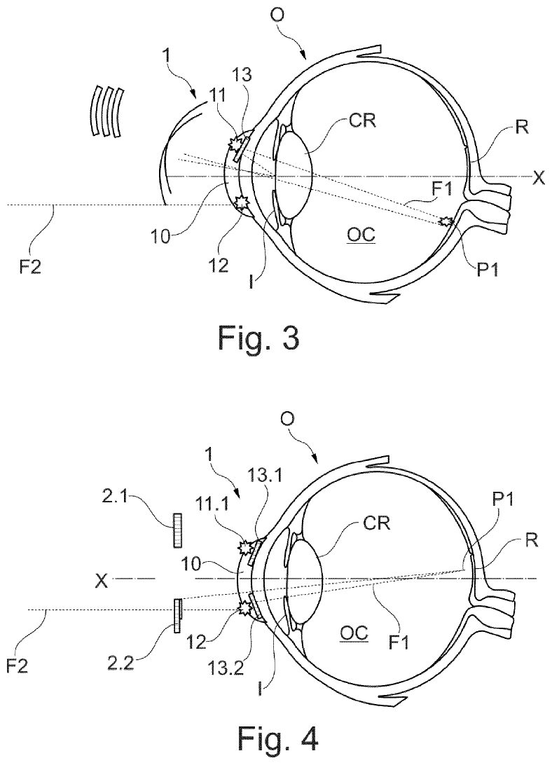

FIG. 3 illustrates one variant of interferometric measurement in which two separate source spots P1, P2 are again created but, unlike FIG. 2, one of the source spots is created on the crystalline lens CR of the eye, the other of the source spots again being created on the retina R of the eye. The interference pattern is therefore between light reflected on the one hand by the source spot on the crystalline lens and on the other hand from the retina R.

FIGS. 4 and 4A illustrate a second embodiment in which refractive error is measured not by interferometry but based on the recording of a multitude of beams sent and reflected sequentially by the retina R. Thus, a plurality of VCSELs each associated with one diffractive optical element 13.1, 13.2 . . . is arranged in the membrane 10 of the lens, in such a way as to be distributed around the eye. A set of beams F1 is sent sequentially to the retina R from the VCSELs and each diffracted by one of the diffractive optical elements. Each beam creates its own source spot on the retina R. The deflection of the sequential beams is recorded with a set of PSDs 2.1, 2.2 . . . placed around the eye. Based on knowledge of the alignment of the eye obtained through the beam F1 and this measurement of deflection of the beams, the refractive error of the eye is determined.

FIG. 5 illustrates a third embodiment in which refractive error is measured not by interferometry, nor based on a multitude of beams sent and reflected sequentially by the retina R, but by holographic analysis. Thus, the diffractive optical element 13 associated with the VCSEL 11 is a hologram, so as to project a holographic pattern onto the crystalline lens R. Image analysis of the deformation of the pattern by a camera 20 makes it possible to estimate the curvature of the crystalline lens R. It is advantageously possible to produce and analyze the deformation of the holographic pattern as described in the patent application FR3106419A1. On the basis of knowledge of the alignment of the eye obtained through the beam F1 and of this estimation of the curvature of the crystalline lens, the accommodation of the eye is determined.

In the case where the contact lens according to the invention incorporates a flexible battery for powering all the electronic/optoelectronic components, a system for recharging this battery by magnetic induction is advantageously provided. Thus, preferably an antenna taking the form of an induction coil 14 connected to a rectifier is encapsulated in a contact lens 1.

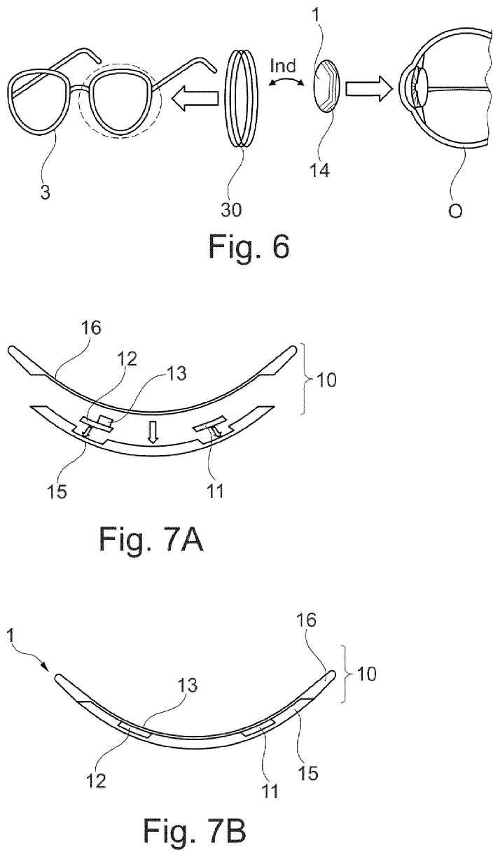

One advantageous example of a recharging system is shown in FIG. 6: an induction antenna 30 is integrated into a spectacle frame 3, which preferably bears the PSDs. The antenna 30 transfers energy via magnetic coupling to the antenna 14 of the contact lens 1, which may be in place on the eye O of an individual during recharging by magnetic induction. Reference may be made to publication [2] for further details.

FIGS. 7A and 7B illustrate certain steps of a method for producing a contact lens according to the invention, of the scleral type.

The membrane 10 here consists of two films 15, 16 of transparent polymer, a hydrogel for example.

Each of the two films 15, 16 is first of all shaped as usual.

Next, all the electronics, with the possible exception of the antenna for collecting energy by induction, are placed on the interior face of the exterior film 15.

Thus, the electronics, including the sources of illumination 11, 12 and, where appropriate, the diffractive optical elements 13, are perfectly positioned within the film 16.

Once this positioning has been carried out, the two films 15, 16 made of transparent polymer are sealed together, for example via UV adhesive.

Thus, all the electronic or optoelectronic components are perfectly positioned and encapsulated between the two films 15, 16.

Other variants and improvements may be made without however departing from the scope of the invention.

In the embodiment where refractive error is measured based on interference of light from source spots on the retina, it is possible to envisage having one (FIG. 1), two (FIG. 2) or a multitude of source spots.

As regards the light beam directed toward the exterior, it may be singular or multiple if a diffractive optical element with which the beam is associated is added.

### LIST OF CITED REFERENCES

[1] C. Stephen, A. Musgrave and F. Fang in the article entitled “Contact Lens Materials: A Materials Science Perspective”, Materials, vol. 14, 261, January 2019.
[2] Y.-J. Kim et al., “Eyeglasses-powered, contact lens-like platform with high power transfer efficiency”, Biomedical Microdevices, vol. 17, no. 4, July 2015.

1. A contact lens intended to be worn by an eye of the individual, to measure refractive error and/or the accommodation of the eye, comprising:
a membrane configured to transparently cover the pupil and to cover the iris of the eye;
at least one source of illumination encapsulated in the membrane, the one or more sources of illumination being configured to emit two cones or beams of light the divergence of which is controlled with respect to the axis of the lens, one of the two beams being intended to be directed toward the interior of the membrane in the direction of the crystalline lens and/or retina of the eye so as to create a source spot on the retina, while the other of the two beams is intended to be directed toward the exterior of the membrane in a direction away from the eye.

2. The contact lens as claimed in claim 1, comprising at least two separate sources of illumination namely one for emitting the beam intended to be directed toward the interior of the membrane in the direction of the crystalline lens and/or retina of the eye and the other for emitting the beam intended to be directed toward the exterior of the membrane in a direction away from the eye.

3. The contact lens as claimed in claim 1, comprising at least one diffractive optical element, encapsulated in the membrane and configured to receive the light beam emitted by the one or more sources of illumination that is directed toward the interior of the membrane and to diffract said beam in the direction of the crystalline lens and/or retina of the eye and to create at least at least two separate source spots on the retina so as to generate interference between the beams reflected by the retina.

4. The contact lens as claimed in claim 1, comprising at least one other diffractive optical element, encapsulated in the membrane and configured to receive the light beam emitted by the one or more sources of illumination that is directed toward the exterior of the membrane and to collimate said beam or project a target or reticle, so as to allow the orientation of the gaze, to be computed or one or more detectors arranged on the periphery of the eye to be addressed.

5. The contact lens as claimed in claim 1, the sources of illumination emitting in the infrared.

6. The contact lens as claimed in claim 1, the one or more sources of illumination being one or more light-emitting diodes (LEDs), or one or more vertical-cavity surface-emitting lasers (VCSELs) or one or more edge-emitting laser diodes.

7. The contact lens as claimed in claim 6, wherein the one or more LEDs or VCSELs are each provided with an optical device for shaping their beams.

8. The contact lens as claimed in claim 1, further comprising:
at least one interface for collecting and supplying the sources of illumination with electrical energy, from the exterior of the lens; and
at least one electronic circuit configured to activate the sources via the interface.

9. The contact lens as claimed in claim 8, further comprising a battery encapsulated in the membrane and connected to the interface, the battery being configured to be recharged via the interface and to supply the sources of illumination and/or optoelectronic functions associated with the sources of illumination electrically, the electronic circuit being configured to activate the sources using the battery.

10. An autorefractor comprising:
at least one contact lens as claimed in claim 1;
a carrier, intended to be positioned stationary with respect to the face of the individual;
at least one detector that is securely fastened to the carrier, the one or more detectors being configured to detect the position of the beam of illumination of the contact lens that is directed toward the exterior so as to extract therefrom the angle of deflection with respect to normal of the gaze;
at least one sensor which is securely fastened to the carrier, and which forms part of a refractometer configured to locate the wavefront of the beam reflected by the retina and/or crystalline lens and refracted by the eye so as to measure the refractive error of the eye given the angle of deflection measured by the detector.

11. The autorefractor as claimed in claim 10, the detector being a position sensitive detector or a camera.

12. The autorefractor as claimed in claim 10, the refractometer being configured to operate as an interferometer configured to measure interference between at least one beam reflected by the retina and refracted by the eye and another illumination beam reflected or by the retina.

13. The autorefractor as claimed in claim 12, the lens comprising a VCSEL by way of source of illumination, the interferometer being configured to perform back-injection laser interferometry between the beam in the interior of the cavity of the VCSEL and a beam reflected by the retina and refracted by the eye.

14. The autorefractor as claimed in claim 12, the lens comprising a VCSEL by way of source of illumination and a diffractive optical element, the interferometer being configured to measure interference between at least two beams that are diffracted by the diffractive optical element from the same beam emitted by the VCSEL, reflected by the retina and refracted by the eye.

15. The autorefractor as claimed in claim 12, the lens comprising a VCSEL by way of source of illumination and a diffractive optical element, the interferometer being configured to measure interference between at least two beams that are diffracted by the diffractive optical element from the same beam emitted by the VCSEL, one being reflected by the retina and refracted by the eye and the other being reflected by the crystalline lens and refracted by the eye.

16. The autorefractor as claimed in claim 10, comprising, as part of the refractometer, a plurality of detectors that are securely fastened to the carrier and arranged to be distributed around the eye, the lens comprising a plurality of VCSELs by way of source of illumination and a plurality of diffractive optical elements each associated with one of the VCSELs, or a single VCSEL associated with at least one optical component generating a plurality of light beams directed toward said detectors, the refractometer being configured to measure the deflection of the beams emitted sequentially by each of the VSCELs, diffracted by each of the diffractive optical elements, reflected by the retina and refracted by the eye.

17. The autorefractor as claimed in claim 10, comprising, as part of the refractometer, a camera that is securely fastened to the carrier, the lens comprising a VCSEL by way of source of illumination and a diffractive optical element taking the form of a hologram, the camera being configured to analyze the deformation of the pattern of the hologram, reflected by the crystalline lens.

18. The autorefractor as claimed in claim 10, the carrier being a mount, intended to be worn on the face of the individual.

19. The autorefractor as claimed in claim 10, comprising a single PSD, intended to be arranged facing the eye, the PSD being transparent in the visible and sensitive in the near infrared , the one or more sources of illumination of the contact lens emitting in the near infrared.

20. The autorefractor as claimed in claim 10, comprising two PSDs, intended to be arranged on the periphery of the eye, substantially in a plane facing the eye, so as to cover the range of angular variation in position of the eye, the PSDs being arranged so as not to obstruct the vision of the individual.
